(19) United States
(12) Patent Application Publication
(10) Pub. No.: US 2011/0150781 A1
(43) Pub. Date: Jun. 23, 2011

(54) TISSUE KALLIKREIN FOR THE TREATMENT OF PARKINSON'S DISEASE

(75) Inventors: Matthew L. Charles, Winnipeg (CA); Mark Williams, Winninpeg (CA)

(73) Assignee: DiaMedica Inc., Manitoba (CA)

(21) Appl. No.: 13/055,660

(22) PCT Filed: Jul. 24, 2009
(86) PCT No.: PCT/CA2009/001051

§ 371 Date: Mar. 4, 2011

Related U.S. Application Data
(60) Provisional application No. 61/083,650, filed on Jul. 25, 2008.

(51) Int. Cl. A61K 38/48; A61P 25/16; A61P 25/00; A61P 25/24

(52) U.S. Cl. 424/43; 424/94.64

(57) ABSTRACT

The invention relates to methods of treating Parkinson's disease, dementia with Lewy bodies, and conditions associated with Parkinson's disease and dementia with Lewy bodies. Methods include administering a therapeutically effective amount of tissue kallikrein, variants or active fragments thereof.

### RELATED APPLICATIONS

This application claims priority to U.S. patent application 60/083,650, filed Jul. 25, 2008, which is incorporated herein in its entirety.

### FIELD OF THE INVENTION

The present invention relates to methods of treating Parkinson's disease, dementia with Lewy Bodies, and other conditions associated therewith.

### BACKGROUND OF THE INVENTION

Parkinson's disease (PD) is a degenerative condition of the central nervous system and the second most common neurodegenerative disease. PD is known to affect individuals over 60 and 80 years of age at rates of >1% and up to 4%, respectively (Schapira et al., Lancet Neurol, 2008, 7: 97-109). PD is typically characterized by the loss of dopaminergic neurons in particular regions of the brain, namely the substantia nigra pars compacta (SNc) (Marras et al., Neurology, 2008, 70(21):1996-2003).

This hindered neurotransmission results in tremor, bradykinesia, and muscular rigidity (Lee, J Mol Neurosci, 2008, 34: 17-22). PD and dementia with Lewy bodies (DLB) can be characterized by the presence of intracytoplasmic inclusions known as Lewy bodies (LB). LB are seen to have a somewhat filamentous structure in which immunohistochemical analysis has revealed that α-synuclein (α-Syn) may be the major component of LB in PD and DLB (Suh et al., Pharmacological Reviews, 2002, 54(3): 469-525). Lewy neurites are similar structures to Lewy bodies and are composed of α-Syn. However, lewy neurites are localized in the axon or dendrite projections of a neuron. These lewy neurites can progress to other regions of the brain and may be responsible for secondary symptoms of PD. Secondary symptoms of PD include depression, dementia, insomnia, autonomic and/or sensory dysfunction, among others (Lee, 2008).

Dementia with Lewy Bodies (DLB) is the second most common form of dementia (Neef, et al., Am Fam Physician, 2006, 73:1223-9). At least 5% of adults 85 years or older have DLB, which is characterized by the presence of LB in the subcortical and cortical (frontotemporal) regions of the brain. LB are composed of abnormal aggregations of α-Syn causing neurodegeneration. The clinical features of DLB include dementia (executive function deficit, visuospatial impairment), delirium, visual hallucinations, parkinsonism (bradykinesia, rigidity, tremors), and depression. Like PD, therapies to treat symptoms include regulation of dopamine levels to improve mobility of DLB patients and administration of cholinesterase inhibitors to treat delirium and visual hallucination symptoms.

PD and DLB present a huge strain on the health care system financially due to therapeutic expenditures for home care and hospitalization. Individuals affected by these diseases often require constant care towards the end of their lives. A variety of therapies to treat the symptoms of PD and DLB are available today, however decreased levels of α-Syn which comprise the Lewy bodies (disease modifying therapy) is thought to be the most effective means for disease management and eradication.

### SUMMARY OF THE INVENTION

The present invention includes methods of treating Parkinson's disease (PD), dementia with Lewy Bodies, and associated conditions, comprising administering a therapeutically effective dose of tissue kallikrein (KLKI), variants, or active fragments thereof.

In an embodiment, associated conditions can be Parkinson's plus syndromes or synucleinopathies, or a combination thereof.

In another aspect, the invention includes a method of improving cleavage of α-synuclein fibrils by administering KLK1, or a variant or an active fragment thereof.

In another aspect, the invention includes a method the breakdown of α-synuclein fibrils by administering KLK1, or a variant or an active fragment thereof.

In another aspect, the invention includes a method of improving neurovasculature by administering KLK1 or a variant or an active fragment thereof.

In another aspect, the invention includes a method of improving oxygen uptake to the brain by administering KLK1, or a variant or an active fragment thereof.

In another aspect, the invention includes a method improving blood flow to the brain by administering KLK1, or a variant or an active fragment thereof.

In another aspect, the present invention includes improved glucose uptake by the brain by administering KLK1, or a variant or an active fragments thereof.

In another aspect, the invention includes a method of improving dopaminergic levels in the brain by administering KLK1, or a variant or an active fragment thereof.

In another aspect of the present invention, tissue kallikrein, or a variant or an active fragment thereof, can be administered orally. Oral administration may be an enteral administration, such as a liquid, pill, or capsule to be swallowed.

In another aspect of the present invention, tissue kallikrein, or a variant or an active fragment thereof, can be administered intranasally.

In a further aspect of the present invention, an oral therapeutic dose can be a maximum dose range of about 0.001 to about 1000 International Units (IU) per day.

In a further aspect of the present invention, a nasal therapeutic dose is a maximum dose of about 0.001 to about 5000 IU per day.

Another aspect of the present invention includes a method comprising administing 1) KLK1, or a variant or an active fragment thereof, and 2) an additional therapeutic compound useful in treating PD. A PD therapeutic compound includes, but is not limited to, an anticholinergic agent, an antiinfective agent, a catechol-O-methyl (COMT) transferase, a dopamine agonist, a monoamine oxidase type B (MAO-B) inhibitor, a neurological agent, a nutritional supplement, a psychotrophic agent, or an antidepressant, or a combination thereof.

A further aspect of the invention wherein an anticholinergic agent that can be benztropine, orphenadrine, procyclidine, or trihexyphenidyl, or a combination thereof.

A further aspect of the invention wherein an antiinfective agent that can be amantadine.

A further aspect of the invention wherein a catechol-O-methyl (COMT) transferase can be carbidopa, entacapone, levodopa or tolcapone, or a combination thereof.

A further aspect of the invention includes a dopamine agonist that can be apomorphine, bromocriptine, cabergoline, pergolide, pramipexole, or ropinirole, or a combination thereof.

A further aspect of the invention includes a monoamine oxidase type B (MAO-B) inhibitor that can be, rasagiline or selegiline, or a combination thereof.

A further aspect of the invention includes a neurological agent that can be brasofensine (investigational), istradefylline (investigational) or leteprinim, or a combination thereof.

A further aspect of the invention includes a nutritional supplement that can be co-enzyme Q-10 and ubiquinone or creatine, or a combination thereof.

A further aspect of the invention includes a psychotrophic agent that can be diphenhydramine.

A further aspect of the invention includes an antidepressant that can be a selective serotonin reuptake inhibitor (SSRI), a tricyclic antidepressant, or another antidepressant drug, or a combination thereof.

A further aspect of the invention includes a selective serotonin reuptake inhibitor (SSRI) that can be citalopram, fluoxetine, paroxetine, or sertaline, or a combination thereof.

A further aspect of the invention includes a tricyclic antidepressant that can be amitriptyline, imipramine, lofepramine, or nortriptyline, or a combination thereof.

A further aspect of the invention includes another antidepressant drug that can be mirtrazapine, moclobemide, phenelzine, or venlafaxine, or a combination thereof.

Another embodiment of the present invention includes a method comprising deep brain stimulation device and administering tissue kallikrein, or a variant or active fragment thereof.

In a further embodiment of the invention, a method of treating PD or DLB may be assessed and monitored by the use of standardized scales.

In a further embodiment of the invention, standardized scales include, but are not limited to, the Modified Hoehn & Yahr Scale, Unified Parkinson's Disease Rating Scale (UPDRS), Schwab and England Scale, or Parkinson's Disease Questionnaire (PDQ 39), or a combination thereof.

Another embodiment of the present invention includes a composition formulated for oral administration comprising about 0.001 to about 1000 IU of KLKI, or a variant or an active fragment thereof, optionally further comprising a pharmaceutically acceptable excipient, and optionally further comprising an additional therapeutic compound as described above.

Another embodiment of the present invention includes a composition formulated for intranasal administration comprising about 0.001 to about 5000 IU of KLKI, or a variant or an active fragment thereof, optionally comprising a pharmaceutically acceptable excipient.

In further embodiments of the invention, a therapeutically effective amount of tissue kallikrein, or a variant or active fragment thereof is administered intranasally.

In further embodiments of the invention, a therapeutically effective dose is about 0.001 to about 5000 International Units (IU) dosage frequency.

In further embodiments of the invention, a therapeutically effective amount of tissue kallikrein, or a variant or active fragment thereof is administered orally.

In further embodiments of the invention, a therapeutically effective of tissue kallikrein, or a variant or active fragment thereof is about 0.001 to about 1000 IU per day.

### BRIEF DESCRIPTION OF THE FIGURES

FIG. 1 shows that treatment with KLK1 leads to significant degradation of α-synuclein. After 24 hour incubation at 37° C., samples were assayed by western blot and stained with N-terminal a-synuclein antibody (ab21975). Lane 1 is 100 nM KLK1+2.5 μM α-synuclein, lane 2 is 100 nM KLK1 alone, lane 3 is 2.5 μM α-synuclein alone, and lane 4 is protein marker standard (kDa).

FIG. 2 shows that treatment with KLK1 leads to significant degradation of a-synuclein. After 24 hour incubation at 37° C., samples were assayed by western blot and stained with C-terminal α-synuclein antibody (ab6162). Lane 1 is 100 nM KLK1+2.5 μM α-synuclein, lane 2 is 100 nM KLK1 alone, lane 3 is 2.5 μM α-synuclein alone, and lane 4 is protein marker standard (kDa).

FIG. 3 shows that treatment with KLK1 leads to significant degradation of α-synuclein 1-95. After 3 hours of incubation at 37° C., samples were assayed by western blot with α-synuclein NAC domain antibody (5C2). Lane 1 is 1 mM KLK1 alone, lane 2 is 250 μg/mla-synuclein 1-95 alone, lane 3 is 1 mM KLK1 and 250 μg/mla-synuclein 1-95, and lane 4 is protein marker standard (kDa).

### DETAILED DESCRIPTION

### Definitions

“Tissue kallikrein” or “KLK1” is a serine protease that is primarily noted for its role in controlling hypertension through its cleavage of kininogen into lysyl-bradykinin (kallidin) (Yousef et al., Endocrine Rev. 2001; 22: 184-204). As there are a large number of enzymes in the KLK family, the inventors believe that KLK1 appears to be a ubiquitous or multiple target acting enzyme, in addition to its recognized role in hypertension regulation and as such may specifically play an important role in treating Parkinson's disease. As used herein, the term “tissue kallikrein” is synonymous with the following terms: callicrein, glumorin, padreatin, padutin, kallidinogenase, bradykininogenase, pancreatic kallikrein, onokrein P, dilminal D, depot-Padutin, urokallikrein, or urinary kallikrein.

Tissue kallikrein polypeptide can have the following sequence:

NP_001001911 GI:50054435 Sus scrofa
1-17 signal peptide
18-24 propeptide
25-263 mature peptide
>gi|50054435|ref|NP_001001911.1| kallikrein 1
[Suss crofa]
(SEQ ID NO: 1)
MWSLVMRLALSLAGTGAAPPIQSRIIGGRECEKDSHPWQVAIYHYSSFQ
CGGVLVDPKWVLTAAHCKNDNYQVWLGRHNLFENEVTAQFFGVTADFPH
PGFNLSLLKNHTKADGKDYSHDLMLLRLQSPAKITDAVKVLELPTQEPE
LGSTCQASGWGSIEPGPDDFEFPDEIQCVELTLLQNTFCADAHPDKVTE
SMLCAGYLPGGKDTCMGDSGGPLICNGMWQGITSWGHTPCGSANKPSIY
TKLIFYLDWINDTITENP

Another embodiment is the human tissue kallikrein polypeptide, which has the following sequence:

NP_002248 GI:4504875 Homo sapiens
1-18 signal peptide
19-24 propeptide
25-262 mature peptide
>gi|4504875|ref|NP_002248.1| kallikrein 1
preproprotein [Homo sapiens]
(SEQ ID NO: 2)
MWFLVLCLALSLGGTGAAPPIQSRIVGGWECEQHSQPWQAALYHFSTFQ
CGGILVHRQWVLTAAHCISDNYQLWLGRHNLFDDENTAQFVHVSESFPH
PGFNMSLLENHTRQADEDYSHDLMLLRLTEPADTITDAVKVVELPTEEP
EVGSTCLASGWGSIEPENFSFPDDLQCVDLKILPNDECKKAHVQKVTDF
MLCVGHLEGGKDTCVGDSGGPLMCDGVLQGVTSWGYVPCGTPNKPSVAV
RVLSYVKWIEDTIAENS

The term “active fragment” refers to smaller portions of a KLK1 polypeptide that retain the serine protease activity of a full-length KLK1 polypeptide.

A “variant” or “mutant” of a starting or reference polypeptide is a polypeptide that 1) has an amino acid sequence different from that of the starting or reference polypeptide and 2) was derived from the starting or reference polypeptide through either natural or artificial (manmade) mutagenesis. Such variants include, for example, deletions from, and/or insertions into and/or substitutions of, residues within the amino acid sequence of the polypeptide of interest. A variant amino acid, in this context, refers to an amino acid different from the amino acid at the corresponding position in a starting or reference polypeptide sequence. Any combination of deletion, insertion, and substitution may be made to arrive at the final variant or mutant construct, provided that the final construct possesses the desired functional characteristics. Amino acid changes may also alter post-translational processes of the polypeptide, such as changing the number or position of glycosylation sites. Methods for generating amino acid sequence variants of polypeptides are described in U.S. Pat. No. 5,534,615, expressly incorporated herein by reference.

A “wild type” or “reference” sequence or the sequence of a “wild type” or “reference” protein/polypeptide maybe the sequence from which variant polypeptides are derived through the introduction of mutations. In general, the “wild type” sequence for a given protein is the sequence that is most common in nature. Similarly, a “wild type” gene sequence is the sequence for that gene which is most commonly found in nature. Mutations may be introduced into a “wild type” gene (and thus the protein it encodes) either through natural processes or through man induced means. The products of such processes are “variant” or “mutant” forms of the original “wild type” protein or gene.

“Percent (%) amino acid sequence identity” with respect to the polypeptides identified herein is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. The ALIGN-2 program is publicly available through Genentech, Inc., South San Francisco, Calif.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y,

where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Percent (%) nucleic acid sequence identity” is defined as the percentage of nucleotides in a candidate sequence that are identical with the nucleotides in a reference polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z,

where W is the number of nucleotides scored as identical matches by the sequence alignment program in that program's alignment of C and D, and where Z is the total number of nucleotides in D. It will he appreciated that where the length of nucleic acid sequence C is not equal to the length of nucleic acid sequence D, the % nucleic acid sequence identity of C to D will not equal the % nucleic acid sequence identity of D to C.

The term “amino acid” is used in its broadest sense and is meant to include the naturally occurring L α-amino acids or residues. The commonly used one and three letter abbreviations for naturally occurring amino acids are used herein (Lehninger, A. L., Biochemistry, 2d ed., pp. 71-92, (1975), Worth Publishers, New York). The term includes all D-amino acids as well as chemically modified amino acids such as amino acid analogs, naturally occurring amino acids that are not usually incorporated into proteins such as Norleucine, and chemically synthesized compounds having properties known in the art to be characteristic of an amino acid. For example, analogs or mimetics of phenylalanine or proline, which allow the same conformational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio, In: The Peptides: Analysis, Synthesis, Biology, Gross and Meiehofer, Eds., Vol. 5 p 341, Academic Press, Inc, N.Y. 1983, which is incorporated herein by reference.

The term “protein” has an amino acid sequence that is longer than a peptide. A “peptide” contains 2 to about 50 amino acid residues. The term “polypeptide” includes proteins and peptides. Examples of proteins include, but are not limited to, antibodies, enzymes, lectins and receptors; lipoproteins and lipopolypeptides; and glycoproteins and glycopolypeptides.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. Preferably, the two portions of the polypeptide are obtained from heterologous or different polypeptides.

“α-synuclein (αsyn or α-Syn)” refers to a 140 amino acid protein that is found in elevated concentrations in the neocortex, hippocampus and substantia nigra, mainly at the synapse, all of which are seen to be altered in the preliminary stages of PD (Suh et al., 2002). Localization of α-Syn suggests a role in the regulation of dopamine release, storage, uptake, and synthesis. α-Syn is known to readily undergo post-translational phosphorylation and can be observed at such elevated levels of phosphorylation in PD and DLB (Suh et al., 2002). Misfolding, oligomerization, and fibrillization of α-Syn are believed to be involved in PD and related disorders progression. α-Syn is highly susceptible to aggregation due to its hydrophobic amino acid portion (Suh et al., 2002), resulting in the formation of intracytoplasmic Lewy bodies. Lewy Bodies have been noted to be enriched with α-Syn, lacking their C-terminus, a form which is noted for its greater rate of aggregation as the C-terminal protection of the aggregation region of α-Syn is absent. Additionally, mutations within the α-Syn protein sequence (e.g., A30P, A53T) also lead to increased aggregation of α-Syn. The formation of Lewy bodies via α-Syn aggregation leads to the generation of reactive oxidative species, a key factor responsible for the death of dopaminergic neurons in the SNc (Junn, et al, Neurosci Lett. 2002, 320;146-150). α-Syn is also able to interact with proteins of the Erk cascade and protein kinase C, which may be involved in a mechanism of neuronal cell death (Suh, 2002). The toxicity of α-Syn beyond the intracytoplasmic space of neurons, has been shown with extracellular forms of α-Syn (fibril and oligomer) being secreted by neurons (Lee, 2008). Extracellular α-Syn may exert its toxic effects through either inserting into the plasma membrane of neurons or activating microglial cells which generate free radicals. α-Syn's oligomeric intermediate form is thought to be the most toxic α-Syn species (Tetzlaff et al., J. Biol. Chem., 2008, 283:17962-17968).

The term “Parkinson-plus syndromes”, as used herein, refers to a group of conditions manifested by the classical features of PD (known as parkinsonism which includes tremor, bradykinesia and muscular rigidity) with additional features which distinguish them from simple idiopathic PD. These conditions include, but are not limited to, Multiple System Atrophy (MSA), Progressive Supranuclear Palsy (PSP) or Corticobasal Degeneration (CBD). These conditions, among others, may also be grouped together as “synucleinopathies” as used herein, which refers to conditions which share common pathological aggregates of α-Syn in selected population of neuronal or glial cells of the brain.

The term “therapeutically effective amount” refers to an amount of a composition of this invention effective to “alleviate” or “treat” a disease or disorder in a subject or mammal. Generally, alleviation or treatment of a disease or disorder involves the lessening of one or more symptoms or medical problems associated with the disease or disorder. In some embodiments, it is an amount that improves neurovasculature, oxygen uptake, blood flow, glucose uptake, dopaminergic levels, or cleavage of fibrils, breakdown of fibrils or a combination thereof.

The terms “treatment” and “treating” refer to inhibiting, alleviating, and healing Parkinson's disease, conditions or symptoms thereof. “Treating” or “treatment” refers to both therapeutic treatment and prophylactic or preventative measures, wherein the object is to prevent or slow down (lessen) the targeted pathologic condition or disorder. Treatment can be carried out by administering a therapeutically effective amount of at least one compound of the invention, and assessed using standardized scales as described herein.

The term “improving neurovasculature” refers to an increase of blood vessel density or increased nutrient delivery to the brain through the blood vessel network. Use of high resolution magnetic resonance imaging (MRI) allows for development of a three dimensional (3D) vascular network map of an imaged brain. Use of endogenous blood oxygenation level-dependent contrast and exogenous contrast agent allows for visualization of artery and vein structures within a 3D image (Bolan et. al, 2006). Comparison of a vascular network before treatment, during treatment, and after treatment allows for assessment of improved neurovasculature for a particular PD or DLB patient undergoing treatment. An “increase” refers to a greater blood vessel density or greater nutrient delivery to the brain in a patient after treatment compared to a blood vessel density or nutrient delivery in the patient before treatment.

The term “improving oxygen uptake” refers to the increased delivery of oxygen to the brain and cells of the brain while the term “improving blood flow” refers to an increase of blood volume circulating through the brain. Use of functional MRI allows for visualization of blood flow in the brain (Davis et. al, 1998). An area of brain that undergoes activity requires oxygen to aid in the metabolism of glucose for energy. This is achieved by a large increase in blood flow so that a diffusion limitation of oxygen is overcome and is supplied in plentiful amounts to active brain tissue. This increase in blood flow and accompanying increase in oxygen is detected through changes in endogenous blood oxygenation level-dependent contrast by functional MRI (Nandhagopal, et al. Neurology 2008, 70:1478-1488). Increased signal is then used to derive the increase in blood flow, oxygen uptake, and metabolism. By mapping areas of blood flow and oxygen uptake deficiencies in the brain of a PD or DLB patient, improvement can be assessed during and after treatment using age matched non-PD or non-DLB patient as a control. An “increase” refers to greater oxygen uptake or blood flow in a patient after treatment compared to oxygen uptake or blood flow in the patient before treatment.

The term “improved glucose uptake” refers to an enhanced ability of the brain to utilize glucose from the blood stream. In PD and DLB there is a reduction of glucose uptake and metabolism by cells of the brain (hypometabolism); this marker of disease onset is determined by the use of PET imaging of the brain with fluorine labeled glucose contrast agent (FDG-PET). By comparing images generated by this method before, during, and after treatment, an improvement in glucose uptake in areas of the brain in a PD or DLB patient previously displaying a reduction of glucose uptake can be assessed while using age-matched non-PD or DLB control subjects.

The term “improving dopaminergic levels” refers to an enhanced ability of the brain to take up L-3,4-dihydroxyphenylalanine (L-DOPA) and intracellular conversion into dopamine which translates into an increase of dopaminergic neurons in the brain. Uptake of L-DOPA is determined by the use of PET imaging of the brain with fluorine labeled 6-F¹⁸-L-DOPA contrast agent (FD PET) (Nandhagopal, et al. 2008). In early stages of Parkinson's Disease, uptake of L-DOPA may increase to compensate for loss of dopaminergic neurons by an increase of activity L-DOPA conversion to dopamine by remaining dopaminergic neurons. This compensatory mechanism disappears as Parkinson's disease progresses leading to a marked drop in L-DOPA uptake measurements by PET scan. By comparing images generated by this method before, during, and after treatment, an improvement in dopaminergic levels in areas of the brain in a PD or DLB patient previously displaying a reduction of dopaminergic levels can be assessed while using age-matched non-PD or non-DLB control subjects.

As well, for DLB, levels of dopaminergic neurons can be assessed by the level of dopamine transporter detection. Dopamine transporter mediates the re-uptake of dopamine from the synaptic cleft and its level is determined by SPECT imaging of the brain with iodine I¹²³-radiolabeled 2β-carbomethoxy-3β-(4-iodophenyl)-N-(3-fluoropropyl)nortropane (FP-CIT) contrast, agent which has affinity to the transporter.

By comparing images generated by this method before, during, and after treatment, an improvement in dopaminergic levels in areas of the brain in a DLB patient previously displaying a reduction of dopaminergic levels can be assessed while using age-matched non-DLB control subjects.

The term “improving cleavage of α-synuclein fibrils” refers to the enhanced ability to proteolytically digest α-synuclein fibrils.

The term “breakdown of α-synuclein fibrils” refers to the outcome of the proteolytic cleavage of α-synuclein fibrils.

The term “standardized scales”, as used herein, refers to questionnaires and inventories, which may be used to assess an individual with Parkinson's disease or Dementia with Lewy Bodies, and can include, but are not limited to, the Modified Hoehn & Yahr Scale, Unified Parkinson's Disease Rating Scale (UPDRS), Schwab and England Scale, or Parkinson's Disease Questionnaire (PDQ 39).

The term “Modified Hoehn & Yahr Scale”, as used herein, refers to, a scale used to assess the degree of disease progression characterized by a numerical value, Stage 0-V where Stage 0 indicates complete absence of the disease while Stage V indicates the disease in its most progressed form. This scale can be seen in Recent Developments in Parkinson's Disease (Fahn S, Marsden C D, Calne D B, Goldstein M, eds. Recent Developments in Parkinson's Disease, Vol 2. Florham Park, N.J. Macmillan Health Care Information 1987, pp 15 3-163, 293-304) and is available online at http://www.mdvu.org/library/ratingscales/pd/updrs.pdf, accessed Jul. 23, 2008.

The term “Unified Parkinson's Disease Rating Scale” or “UPDRS”, as used herein, refers to a scale used to assess the degree of disease progression by evaluation from a physician as well as patient self evaluation resulting in a point score of 0-199, where 0 indicates complete absence of the disease while 199 indicates the disease in its most progressed form. This scale can be seen in Recent Developments in Parkinson's Disease (Fahn S, Marsden C D, Caine D B, Goldstein M, eds. Recent Developments in Parkinson's Disease, Vol 2. Florham Park, N.J. Macmillan Health Care Information 1987, pp 15 3-163, 293-304) and is available online at http://www.mdvu.org/library/ratingscales/pd/updrs.pdf, accessed Jul. 23, 2008.

The term “Schwab and England Scale” as used herein, refers to, a scale which quantifies a patients ability to perform tasks and their level of independence upon task performance and completion as an indication of disease progression. This scale can be seen in Recent Developments in Parkinson's Disease (Fahn S, Marsden C D, Caine D B, Goldstein M, eds. Recent Developments in Parkinson's Disease, Vol 2. Florham Park, N.J. Macmillan Health Care Information 1987, pp 15 3-163, 293-304) and is available online at http://www.mdvu.org/library/ratingscales/pd/updrs.pdf, accessed Jul. 23, 2008.

The term “Parkinson's Disease Questionnaire” or “PDQ 39”, as used herein, refers to a self-administered questionnaire provided to patients to determine the degree of disease progression where a low score indicates the disease at a mild stage while a higher score indicates a more progressed stage of the disease, as described by Katsarou (Katsarou et al., Quality of Life Research, 2001, 10: 159-163).

Methods of Treating Parkinson's Disease and Dementia with Lewy Bodies

Treatment of PD symptoms primarily focuses on regulating the levels of dopamine by increasing dopamine levels (e.g., levodopa given with carbidopa), the use of dopamine agonists (e.g., Bromocriptine), or by decreasing the level of acetylcholine (e.g., anticholinergics) to compensate for reduced dopamine levels. These therapies along with physiotherapy are used to help improve mobility. Treatment of depression symptoms in those with PD can be managed with tricyclic antidepressants, selective serotonin re-uptake inhibitors, or monoamine oxidase A inhibitors. However, a number of antidepressants, namely D2 antagonist based typical antipsychotics (neuroleptics), should be avoided, as PD symptoms can be made worse.

The present invention provides methods for treating Parkinson's disease, dementia with Lewy Bodies, and conditions associated therewith. One embodiment includes a method of treating Parkinson's disease, and/or dementia with Lewy Bodies by administering a therapeutically effective amount of tissue kallikrein, or variant or active fragment thereof. Tissue kallikrein, or variant or active fragment thereof, can be administered to a mammal, preferably, a human.

Activation of MMP9 from its inactive pro-MMP9 form is thought to occur by the action of extracellular proteases, including KLK1 (Desrivieres et. al, J Cell Physiol, 1993, 157(3): 587-93). Studies have shown the ability of MMP9 to cleave α-Syn at four sites in the NAC region, thereby resulting in depleted α-Syn aggregation and complete breakdown of α-Syn (Sung et al., The Journal of Biological Chemistry, 2005, 280(26):25216-25224). The protease activity of KLK1 is likely responsible for the cleavage of α-Syn either directly or indirectly (by MMP-9 activation).

The 1-methyl-4-phenyl-1,2,3,6-tetrahydropyridine (MPTP) mouse model of PD is directly linked to the upregulation of α-Syn expression and increased levels of hyperphosphorylated Tau protein. MPTP induction of PD results in an abrupt activation of GSK-3β, which is known to play a role in the neurotoxic action of MPTP, resulting in apoptosis and therefore may be a target for disease treatment (Wang et al., Neuro Pharmacology, 2007, 52:1678-1684). Inhibition of GSK-β protected against MPTP mediated cell death of dopamingeric neurons and resulted in an improvement in animal behavior.

The activity of GSK-3β is normally regulated by serine 9 phosphorylation by Akt. Activation of the kinin B₂ receptor signaling pathway by kinin (Yin et al., J Biol Chem, 2005, 280(9): 8022-30) and the NGF-acetylcholine pathway leads to increased GSK-3p phosphorylation (Rylett et al., J Neurosci, 1993, 13(9): 3956-3963)(De Sarno et al., Neurobiol Aging, 2006, 27(3): 413-22.). Both pathways are thought to be mediated by extracellular proteases, including KLK1 (Castro et al., FEBS Lett, 1990, 267(2): 207-12) (Xia et al., Hypertension, 2006, 47(4): 752-61).

Additional routes of activation of the PI3K/Akt/GSK-3β pathway can be achieved by the binding of neurotrophins, such as NGF, to their respective Trk receptors, (Trk A for NGF). Neurotrophins are noted for their ability to provide neuroprotection from apoptosis and neurodegeneration, promote axonal growth and improve neuronal synaptic connectivity (Buckley et al., Schizophr Res, 2007, 94(1-3): 1-11). As well, binding to their appropriate Trk receptor leads to an increase in their production. The binding of NGF to Trk A, leading to Akt activation, can only take place once NGF has been cleaved into its mature form. The precursor form of NGF is post-transitionally modified by KLK1 cleavage into its mature form such that NGF is able to activate the PI3K/Akt/GSK-3β pathway through Trk A. Animal models and patients with PD have shown reduced NGF levels (Lorigados, et al., Brain Res. 2002 952:122-127) and that signaling through NGF can protect from MPTP mediated cell death (Shimoke, et al., J Neurosci Res. 2001 63:402-409).

As such, a method of treating PD or DLB through the administration of KLK1, a variant or active fragment thereof, either orally or via the intranasal route, improves GSK-3β regulation in the brain.

Administration of Tissue kallikrein

Traditional modes of drug administration to treat aliments in the brain include oral as well as intravenous routes of administration. These modes are not always ideal. Oral administration of compounds results in limited bioavailability (solubility, 1^{st} pass liver degradation, blood brain barrier restriction) as well as time release issues with potentially undesirable gastrointestinal side effects. However, Tissue Kallikrein (KLK1) appears able to pass through and bypass the blood-brain-barrier such that it may produce its effects on the brain.

An oral dose of KLKI, variant, or active fragment thereof, can be a dose of about 1 to about 1000 IU per day; about 1 to about 750 IU per day; about 1 to about 500 IU per day; about 1 to about 400 IU per day; about 1 to about 300 IU per day; about 1 to about 250 IU per day; about 1 to about 200 IU per day; about 1 to about 150 IU per day; about 1 to about 100 IU per day; about 1 to about 75 IU per day; about 1 to about 50 IU per day; about 1 to about 50 IU per day; about 1 to about 25 IU per day; about 1 to about 20 IU per day; about 1 to about 15 IU per day; about 1 to about 10 IU per day; about 1 to about 5 IU per day; about 5 to about 1000 IU per day; about 10 to about 1000 IU per day; about 15 to about 1000 IU per day; about 20 to about 1000 IU per day; about 25 to about 1000 IU per day; about 50 to about 1000 IU per day; about 75 to about 1000 IU per day; about 100 to about 1000 IU per day; about 150 to about 1000 IU per day; about 200 to about 1000 IU per day; about 250 to about 1000 IU per day; about 300 to about 1000 IU per day; about 400 to about 1000 IU per day; about 500 to about 1000 IU per day; about 750 to about 1000 IU per day; about 10 to about 100 IU per day; about 10 to about 250 IU per day; about 10 to about 500 IU per day; about 50 to about 250 IU per day; about 50 to about 500 IU per day; about 100 to about 250 IU per day; about 100 to about 500 IU per day; or about 250 to about 750 IU per day.

Intravenous (i.v.) administration may require trained medical professionals, which is time consuming and costly to the health care system and may result in patient compliance issues. Risks associated with intravenous administration are also present, namely infection at the injection site and a variety of safety issues to the patient and the professional administering the dose.

Intranasal administration allows a medicament to be ‘fast acting’ since it is able to reach the brain by a more direct route. Intranasal administration is convenient and virtually eliminates issues of patient compliance as seen with intravenous administration. The cells of the olfactory epithelium are selectively permeable. Proteins such as KLK1 may be able to pass through and may bypass the blood-brain-barrier via the intranasal route, such that it may produce its effects directly on the brain, thereby minimizing peripheral effects as well. This is due to involvement of the olfactory region in the upper portion of the nasal pathway.

A intranasal dose of KLKI, variant, or active fragment thereof, can be a dose of about 1 to about 5000 IU per day; about 1 to about 4000 IU per day; about 1 to about 3000 IU per day; about 1 to about 2500 IU per day; about 1 to about 2000 IU per day; about 1 to about 1000 IU per day; about 1 to about 750 IU per day; about 1 to about 500 IU per day; about 1 to about 400 IU per day; about 1 to about 300 IU per day; about 1 to about 250 IU per day; about 1 to about 200 IU per day; about 1 to about 150 IU per day; about 1 to about 100 IU per day; about 1 to about 75 IU per day; about 1 to about 50 IU per day; about 1 to about 50 IU per day; about 1 to about 25 IU per day; about 1 to about 20 IU per day; about 1 to about 15 IU per day; about 1 to about 10 IU per day; about 1 to about 5 IU per day; about 5 to about 1000 IU per day; about 10 to about 1000 IU per day; about 15 to about 1000 IU per day; about 20 to about 1000 IU per day; about 25 to about 1000 IU per day; about 50 to about 1000 IU per day; about 75 to about 1000 IU per day; about 100 to about 1000 IU per day; about 150 to about 1000 IU per day; about 200 to about 1000 IU per day; about 250 to about 1000 IU per day; about 300 to about 1000 IU per day; about 400 to about 1000 IU per day; about 500 to about 1000 IU per day; about 750 to about 1000 IU per day; about 10 to about 100 IU per day; about 10 to about 250 IU per day; about 10 to about 500 IU per day; about 50 to about 250 IU per day; about 50 to about 500 IU per day; about 100 to about 250 IU per day; about 100 to about 500 IU per day; or about 250 to about 750 IU per day.

Intranasally administered drugs can reach tissues of the brain and spinal cord using an extracellular route through perineural channels. This intranasal administration delivers a drug to the upper third (⅓) of the nasal cavity where the drug is absorbed through nasal mucosa.

There are two possible routes that a substance administered intranasally may follow at the olfactory epithelium. These are said to be intraneuronal and extraneuronal. An intraneuronal route is by uptake of peptides into olfactory neurons where peptides may travel along axons to bypass the blood-brain-barrier. Passage through unique intercellular clefts in epithelia of the olfactory region is an extracellular route that allows peptides to diffuse into the subarachnoid space. An extracellular route is more preferable due to rapid passage time to the brain, avoidance of proteolytic degradation involved in intraneuronal pathways (Born et al., Nat. Neurosci. 2002, 5(6):514-6), and rapid eliciting of biological effects at multiple sites of the brain (Throne et al. Neuroscience, 2004, 127(2): 481-96).

Although oral delivery is possible and advantageous, an other route of administration is intranasal due to more direct delivery of KLK1 to desired sites of action (the brain).

Pharmaceutical compositions may be administered orally or intranasally. Formulations suitable for intranasal administration comprise 0.001 to about 5000 IU per dosage frequency of tissue kallikrein, or a variant or active fragment thereof, and can be, ointments, creams, lotions, pastes, gels, sprays, aerosols, oils and the like. Solutions or suspensions can be applied directly to the nasal cavity by conventional means, for example, with a dropper, pipette or spray. Formulations can be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

Formulations for aerosol administration, particularly to the upper respiratory tract containing the nasal cavity and olfactory region, include intranasal administration. An active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. An aerosol may conveniently also contain a surfactant such as lecithin. A dose of drug may be controlled by a metered valve. Alternatively active ingredients may be provided in a form of a dry powder, for example a powder mix of the compound in a suitable powder base such as lactose, starch, starch derivatives such as hydroxypropylmethyl cellulose and polyvinylpyrrolidine (PVP). A powder carrier will form a gel in the nasal cavity. A powder composition may be presented in unit dose form for example in capsules or cartridges of e.g., gelatine or blister packs from which the powder may be administered by means of an inhaler.

Formulations suitable for oral administration comprise 0.001 to about 1000 IU per dosage frequency of tissue kallikrein, or a variant or active fragment thereof, and can be, solution, tablets, sustained release capsules, enteric coated capsules, orally disintegrating tablets and syrups.

An “effective amount” or a “therapeutically effective amount” refers to a nontoxic but sufficient amount of drug or agent to provide a desired effect. In a combination therapy, an “effective amount” of one component of the combination is an amount of that compound that is effective to provide a desired effect when used in combination with the other components of the combination. An amount that is “effective” will vary from subject to subject, depending on the age and general condition of an individual, a particular active agent or agents, and the like. An appropriate “effective” amount in any individual case may be determined using routine experimentation.

A therapeutically effective amount of a compound of the invention for treating the above-identified diseases or symptoms thereof can be administered prior to, concurrently with, or after the onset of the disease or symptom. A compound of the invention can be administered concurrently with the onset of the disease or symptom. “Concurrent administration” and “concurrently administering” as used herein includes administering a polypeptide of the invention and another therapeutic agent in admixture, such as, for example, in a pharmaceutical composition or in solution, or separately, such as, for example, separate pharmaceutical compositions or solutions administered consecutively, simultaneously, or at different times, but not so distant in time such that the compound of the invention and the other therapeutic agent cannot interact and a lower dosage amount of the active ingredient cannot be administered.

### PD Therapeutic Compounds

Another aspect of the present invention includes a method as herein described further comprising concurrently administering an additional therapeutic compound useful in treating PD or dementia with Lewy bodies. A PD therapeutic compound includes, but is not limited to, an anticholinergic agent, an antiinfective agent, a catechol-O-methyl (COMT) transferase, a dopamine agonist, a monoamine oxidase type B (MAO-B) inhibitor, a neurological agent, a nutritional supplement, a psychotrophic agent, or certain antidepressants, such as tricyclic antidepressants, or a combination thereof.

An anticholinergic agent includes, but is not limited to benztropine, orphenadrine, procyclidine, or trihexyphenidyl, or a combination thereof.

An antiinfective agent includes, but is not limited to amantadine.

A catechol-O-methyl (COMT) transferase, includes, but is not limited to carbidopa, entacapone, levodopa or tolcapone, or a combination thereof.

A dopamine agonist, includes, but is not limited to apomorphine, bromocriptine, cabergoline, pergolide, pramipexole, or ropinirole, or a combination thereof.

A monoamine oxidase type B (MAO-B) inhibitor, includes, but is not limited to rasagiline or selegiline, or a combination thereof.

A neurological agent, includes, but is not limited to brasofensine (investigational), istradefylline (investigational) or leteprinim, or a combination thereof.

A nutritional supplement, includes, but is not limited to co-enzyme Q-10 and ubiquinone or creatine, or a combination thereof.

A psychotrophic agent, includes, but is not limited to diphenhydramine.

An antidepressant, includes, but is not limited to a selective serotonin reuptake inhibitor (SSRI), a tricyclic antidepressant, or another antidepressant drug, or a combination thereof.

A selective serotonin reuptake inhibitor (SSRI), includes, but is not limited to citalopram, fluoxetine, paroxetine, or sertaline, or a combination thereof.

A tricyclic antidepressant, includes, but is not limited to amitriptyline, imipramine, lofepramine, or nortriptyline, or a combination thereof.

Another antidepressant drug, includes, but is not limited to mirtrazapine, moclobemide, phenelzine, or venlafaxine, or a combination thereof.

Another aspect of the present invention includes a method as herein described further comprising deep brain stimulation and adminitering a therapeutically effective dose of tissue kallikrein for treating PD. Deep brain stimulation (DBS) is a surgical treatment in which electrodes (leads connected to the pulse generator) are implanted into areas of the brain responsible for symptoms of PD, for example, the subthalamic nucleus and the globus pallidus interna areas. DBS is commonly referred to as a brain pacemaker as the pulse generator sends electrical impulses deep into the brain along the implanted lead electrodes on a regular basis. DBS is primarily recommended to those who are treatment-resistant to PD drug therapies.

“Treatment” and “treating” refer to preventing, inhibiting, and/or alleviating Parkinson's disease or dementia with Lewy bodies, and related symptoms as well as healing Parkinson's disease or dementia with Lewy bodies, conditions or symptoms affecting mammalian organs and tissues. A composition of the present invention can be administered in a therapeutically effective amount to a patient before, during, and after any above-mentioned condition arises.

### Intranasal Administration

An aspect of the invention includes a composition formulated for intranasal administration comprising about 0.001 to about 5000 IU of KLKI, or a variant or an active fragment thereof, optionally comprising a pharmaceutically acceptable excipient. A composition can be administered to the nasal cavity of a human or other mammal to diseased areas of the brain by means of the olfactory neural pathway. The method may employ a pharmaceutical composition capable of transporting KLK1 to diseased neurons of the brain.

A method of the invention may achieve delivery of compounds to afflicted areas of the brain through transneuronal retrograde and anterograde transport mechanisms. Delivery of neurologic agents to the brain by that transport system may be achieved in several ways. One technique comprises delivering a neurologic agent alone to the nasal cavity. In this instance, chemical characteristics of KLK1 can facilitate its transport to diseased neurons in the brain. Alternatively, KLK1 may be combined with other substances that assist in transporting KLK1 to sites of damaged neurons. Auxiliary substances are capable of delivering KLK1 to peripheral sensory neurons and/or along neural pathways to dysfunctional areas of the brain. Peripheral nerve cells of the olfactory neural pathway can be utilized in order to deliver KLK1 to damaged neurons in those regions of the brain that are connected to the olfactory bulb.

KLK1 can be administered to the nasal cavity alone or in combination with a second therapeutic compound useful in treating PD or DLB. KLK1 can be combined with a carrier and/or other adjuvants to form a pharmaceutical composition. Potential adjuvants include, but are not limited to, GM-1, phosphatidylserine (PS), and emulsifiers such as polysorbate 80. Further supplementary substances include, but are not limited to, lipophilic substances such as gangliosides and phosphatidylserine (PS).

A method of the invention delivers KLK1 to the nasal cavity of a mammal. It is preferred that KLK1 be delivered to the olfactory area in the upper third of the nasal cavity and particularly to the olfactory epithelium in order to promote transport of the agent into the peripheral olfactory neurons rather than the capillaries within the respiratory epithelium. Transport of KLK1 to the brain by means of the nervous system instead of the circulatory system so that KLK1 can be delivered to damaged neurons in the brain.

In one embodiment of the method of the invention, KLK1 can be combined with micelles comprised of lipophilic substances. Such micelles may modify the permeability of the nasal membrane and enhance absorption of the agent. Lipophilic micelles can include gangliosides, particularly GM-1 ganglioside, and phosphatidylserine (PS).

Once KLK1 has crossed the nasal epithelium, the invention further provides for transport of KLK1 along the olfactory neural pathway. KLK1 may be capable of movement within the olfactory system. In particular, neurotrophic and neuritogenic substances have demonstrated ready incorporation into nerve cell membranes and an affinity for nerve cell receptor sites.

To deliver KLK1 to olfactory neurons, KLK1 alone or in combination with other substances as a pharmaceutical composition may be administered to the olfactory area located in the upper third of the nasal cavity. The composition may be dispensed intranasally as a powdered or liquid nasal spray, nose drops, a gel or ointment, through a tube or catheter, by syringe, by packtail, by pledget, or by submucosal infusion.

A pharmaceutical composition for intranasal administration may be formulated as a powder, granules, solution, ointment, cream, aerosol, powder, or drops. A solution may be sterile, isotonic or hypotonic, and otherwise suitable for administration by injection or other means. In addition to KLK1, a solution may contain appropriate adjuvants, buffers, preservatives and salts. Powder or granular forms of a pharmaceutical composition may be combined with a solution and with diluting, dispersing and/or surface active agents. Solutions such as nose drops may contain antioxidants, buffers, and the like.

Intranasal administration methods provide an advantage of the intranasal administration of the medication. The olfactory system provides a direct connection between the outside environment and the brain thus providing quick and ready delivery of KLK1 for treating PD and DLB. Moreover, means of applying a pharmaceutical composition intranasally can be in a variety of forms such as a powder, spray, or nose drops that obviates intravenous or intramuscular injections and simplifies administration of therapeutic medications.

The invention will be described with reference to various specific and preferred embodiments and techniques. However, it should be understood that many variations and modifications may be made while remaining within the spirit and scope of the invention.

### EXAMPLES

### Example 1

### In vitro Cleavage of α-Synuclein by KLK1

### Cleavage Assay

To assay the ability of KLK1 to cleave full length α-synuclein (1-140 AA), recombinant α-synuclein (rPeptide) was treated with porcine pancreas derived KLK1 in PBS solution. The following samples were incubated for 24 hours at 37° C.: 100 nM KLK1+2.5 μM α-synuclein, 100 nM KLK1 alone, and 2.5 μM α-synuclein alone. Each sample of KLK1 contained 50 nM of soybean trypsin inhibitor (Sigma, St. Louis). At the completion of the incubation period, the samples were run on a 15% SDS-PAGE gel which was then assayed by Western Blot on a PVDF membrane. The membrane was incubated in primary antibody (ab6162 or ab21975) diluted 1:5000 in blocking solution, followed by incubation with and alkaline phosphatase-conjugated secondary antibody. The membrane was then developed in a NBT-BCIP solution.

To assay the ability of KLK1 to cleave α-synuclein 1-95 (1-95 AA), recombinant α-synuclein 1-95 (GenWay Biotech) was treated with porcine pancreas derived KLK1 in PBS solution. The following samples were incubated for 3 hours at 37° C.: 1 mM KLK1 alone, 250 μg/ml α-synuclein 1-95 alone, and 1 mM KLK1 and 250 μg/ml α-synuclein 1-95. Each sample of KLK1 contains 100 μM of soybean trypsin inhibitor. At completion of the incubation period, the samples run on a 15% SDS-PAGE gel and which was then assayed by Western Blot on a PVDF membrane. The membrane was incubated in primary antibody (5C2) diluted 1:250 in blocking solution, followed by incubation with and alkaline phosphatase-conjugated secondary antibody. The membrane was then developed with chemiluminscent solution and exposed onto film.

### Analysis of Cleavage by Western Blot

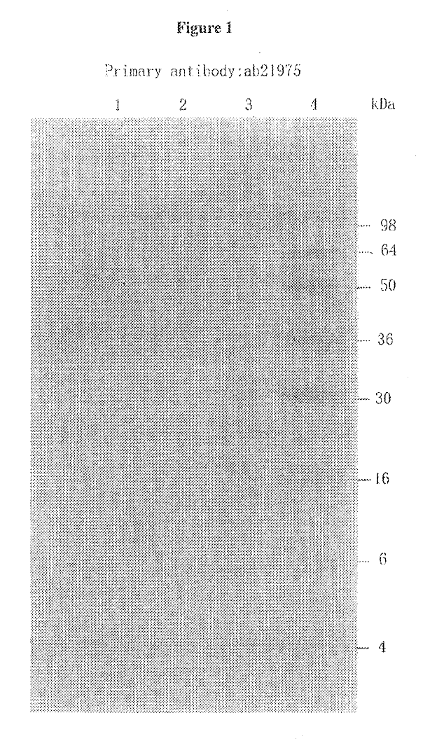

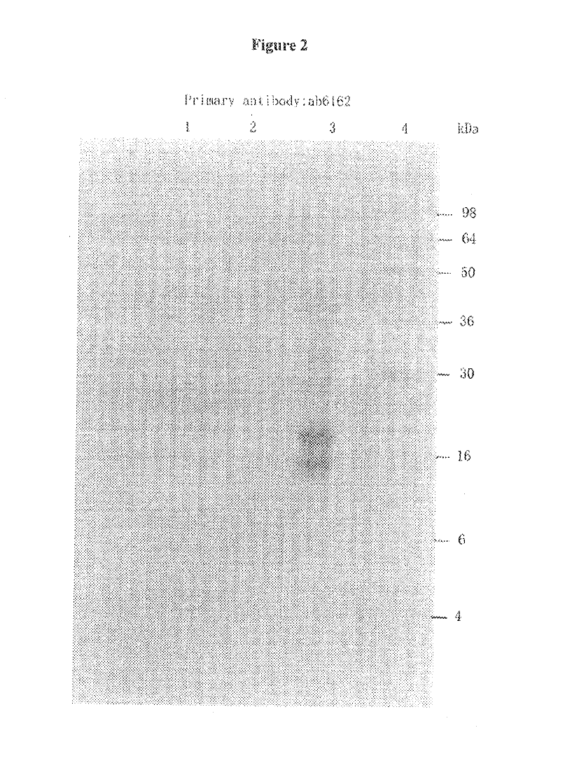

After 24 hours of treatment, all detectable α-synuclein was degraded by KLK1. α-synuclein was not detected after treatment with KLK1 by N-terminal specific (FIG. 1, lane 2) and C-terminal specific (FIG. 2, lane 2) antibodies, however could be detected as being intact when not treated with KLK1 (FIG. 1, lane 3 and FIG. 2, lane3). α-synuclein is the main component of neuronal protein aggregations called Lewy bodies and Lewy neurites. It is believed that these inclusions are neurotoxic and result in the death of doaminergic neurons within the substantia nigra of the brain in those with Parkinson's disease. α-synuclein degradation is disturbed in Parkinson's disease, and therefore treatment with KLK1 may prevent α-synuclein aggregation and formation of Lewy bodies and Lewy neurites.

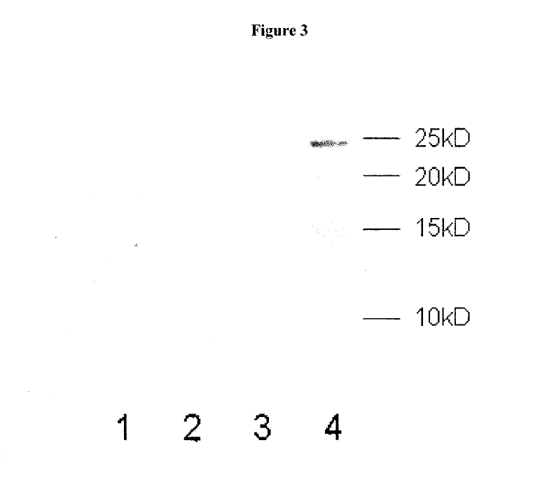

After 3 hours of treatment, all detectable α-synuclein 1-95 was degraded by KLK1. α-synuclein 1-95 was not detected after treatment with KLK1 by the non-13-amyloid component (NAC) specific antibody (FIG. 3, lane 3), but could be detected as being intact when not treated with KLK1 (FIG. 3, lane 2). The NAC region of α-synuclein is believed to be the aggregation domain due to its hydrophobic sequence, leading to the formation of Lewy body and Lewy neurite inclusions. The C-terminal sequence following NAC region appears to play an inhibitory role in preventing α-synuclein aggregation. C-terminal truncations with intact NAC regions of α-synuclein lead to enhanced aggregation, neurotoxicity of dopaminergic neurons and are found within Lewy inclusions of Parkinson's disease (Periquet et al, 2007). α-synuclein 1-95 is a C-terminal truncation with an intact NAC region, yet is completely degraded by KLK1, thereby suggesting KLK1 may be effective treatment against the most toxic form of α-synuclein in Parkinson's disease.

### Example 2

### Inhibition of GSK-313 by KLK1 Leads to Decreased α-Synuclein Expression and Cell Death from MPTP Treatment

The apoptosis of neurons leads to the neurodegeneration seen in Parkinson's disease. MPTP (N-methyl-4-phenyl-1,2,3,6-tetrahydropyridine) is a neurotoxin which imparts its toxic affects through the activation of the kinase GSK-3β which leads to increased expression of α-synuclein protein.

Treatment of Neuronal Cells with MPTP for 48 Hours

Primary mesencephalic neurons isolated from 16-18 day old rat embryos are plated onto 6 well dishes and grown in each well using Neurobasal medium (Invitrogen) supplemented with B27 (2% v/v, Invitrogen), penicillin/streptomycin mixture (10 U mL¹) and 25 μm β-mercaptoethanol at 37° C. and 5% CO₂. 50 μM of MPP⁺ iodide (Sigma, St. Louis, Mo.) is added to each well and allowed to incubate for 48 hours. In addition, KLK1 (0.01 to 100 Units) is added to the MPP⁺ treated wells 24 hours after the addition of the neurotoxin. A positive control is the non-treated well over 48 hours.

Western Blot Analysis after 48 Hours MPTP Treatment

The mesencephalic neurons are washed twice with ice-cold PBS and collected with 200 μl 2× stop solution (500 mM Tris-HCL, pH 6.8, 10% SDS, 100 mM EDTA, 100 mM EGTA, 10% glycerol) containing protease inhibitor mixture (Complete Mini, Roche Diagnostics GmbH, Germany) and the phosphatase inhibitor sodium orthovanadate (1 mM). The samples are resolved by NuPAGE 12% Bis-Tris (Invitrogen, Carlsbad, Calif.) SDS-PAGE gel electrophoresis and followed by electroblotting onto a PVDF membrane in NuPAGE transfer buffer. The membrane is then separately blotted using a rat specific α-Syn antibody (BD Transduction Laboratories, San Jose, Calif., 610786) for detection of full sized α-Syn and rat specific GSK-3β antibody (pY216, BD Transduction Laboratories, 612312). The quantization of the each treatment group lane is obtained by measuring optical density of each band and expressed as a ratio between α-Syn+β-actin, and GSK-β+p-GSK-3βY216, separately. The ratio values obtained for each treatment group lane are then compared to the control group (which is set to 100%).

### Results

MPTP leads to a significant increase in GSK-3β activation (increased pY216) and α-synuclein protein expression compared to the control. The Western blot shows that supplemental treatment of the neuronal cells 24 hours after MPTP treatment with KLK1 leads to a statistically significant decrease in GSK-3β activation (decreased pY216) and α-synuclein protein expression compared to MPTP treatment alone. These data suggest KLK1 can prevent the activation of GSK-3βand increased expression of α-Synuclein by MPTP.

MTT Assay for Cell Viability

After 48 hours of treatment with MPTP, MPTP followed by KLK1 at 24 hours or non-treatment control, the mesencephalic neuron treatment groups are washed twice with D-PBS and then incubated for 2 hours with Neurobasal medium (Invitrogen) supplemented with B27 (2% v/v, Invitrogen), penicillin/streptomycin mixture (10 U mL¹), 25 μm β-mercaptoethanol and 0.5 mg mL⁻¹ of MTT (3-(4,5-dimethylthiazol-2-yl)-2,5-diphenyltetrazolium bromide, Sigma, St. Louis, Mo.) at 37° C. and 5% CO₂. The cells are then washed carefully twice with D-PBS and then the formazan salts are solubilized with pure ethanol. A sample from each treatment group is measured by absorbance at λ=564 nm using a UVN spectrophotometer using ethanol as the blank reference. The control absorbance reading is set to 100% and the other treatments are expressed as % of the control.

### Results

In comparison to the MPTP alone treatment group, the supplementation of KLK1 24 hours after MPTP results in no statistical difference in cell viability compared to the non-treatment control. This result suggests that KLK1 prevents the ability of MPTP to exert its toxicity on neuronal cells, likely through the ability of KLK1 to prevent the activation of GSK-3β and resulting increased α-synuclein expression.

### Example 3

### KLK1 Enhances Cell Viability After Treatment with α-Syn

### Aggregate Preparation:

Various forms of α-Syn (500 μM) (e.g. wild-type, minus C-terminal, A30P, A53T forms) are allowed to aggregate into fibrils by incubation in sterile water solution at 37° C. for three days, while non-aggregated forms are prepared immediately before application to the cultures.

### Cell Treatment:

At embryonic day 14, brain tissue is removed from fetal rats and the cells are plated (3×10⁵ cells per dish) in 96-well dishes (Corning, Corning, N.Y.) precoated with poly-d-lysine (50 mg/ml; Sigma, St. Louis, Mo.). The mesencephalic neurons are grown in minimum essential medium (Gibco Laboratories, Grand Island, N.Y.) supplemented with 15% fetal calf serum (Gibco) and glutamine (2 mM). Cultures are kept at 37° C. in a humidified 5% CO₂.

The cells are treated with aggregated and non-aggregated α-Syn (10, 25, 50, or 100 μM) on every second day for six days with or without KLK1 (0.01 to 100 Units) beginning 24 hours after cell plating. Using the MTT assay for cell viability (as per Example 2) the effect of α-Syn on mesencephalic neuron viability is determined, expressed as percentage of the non-treated control cell viability.

### Results:

The treatment of mesencephalic neurons with α-Syn leads to a significant decrease in cell viability compared to the non-treatment control group. The addition of KLK1 to the treatment with α-Syn leads to a significant increase in cell viability compared to the mesencephalic neurons treated with α-Syn only. This result shows the ability of KLK1 to protect mesencephalic neurons from α-Syn induced neurodegeneration.

### Example 4

### In Vivo KLK1 Mediated Neuroprotection from MPTP Neurotoxin Treatment

MPTP is a neurotoxin which preferentially exerts its toxicity on dopaminergic neurons within the substantia nigra pars compacta (SNc), an area of the brain severely affected by neurodegeneration in Parkinson's disease.

### MPTP Treatment of C58BL/6N Mice

After a one week rest period, 12 week adult male C58BL/6N mice were divided into three treatment groups with 10 mice in each group. In the MPTP alone treatment group, mice are injected intraperitoneally (i.p.) with 30 mg/kg of MPTP (Sigma) daily for five consecutive days. Mice of the KLK1+MPTP treatment group are treated with intranasal administration of KLK1 (0.01 to 100 Units) one day before the start of MPTP i.p. administration and in the following 5 days thirty minutes before MPTP treatment. The control non-treatment group receives injections of saline solution for 5 days consecutively.

### Assessment of Neurodegeneration of the Substantia Nigra Pars Compacta

Seven days following the final injection of MPTP, midbrain slices are stained for tyrosine hydroxylase (TH⁺) activity of dopaminergic neurons by immunohistochemistry using an anti-mouse TH antibody (Chemicon) and DAB staining. TH⁺ cells within the substantia nigra pars compacta (SNc) are counted in a double blind manner determining the mean count of each right and left sides from four sections under a light microscope.

### Results:

In comparison to the saline vehicle treated control group, the MPTP treated group shows a statistically significant decrease in TH⁺ neurons. The KLK1+MPTP treatment group shows a statistically significant higher number of TH⁺ neurons within the SNc compared to MPTP alone group. KLK1 significantly prevents TH⁺ dopaminergic neuron death from MPTP mediated-apoptosis, such that KLK1 prevents neurodegeneration in the SNc, an area of the brain in PD in which neurodegeneration is commonly found.

### Assessment of Behavioural Impairment in MPTP-Treated Mice

10 days following the final injection of MPTP, mice in each treatment group are tested for behavioral impairment in a double blinded manner.

### Pole Test

Mice are placed on a vertical wooden pole (50 cm in length and 1 cm wide) with their head facing upwards. The total time required for a mouse to turn downward and climb to the ground is recorded, with a cut off of 120 sec.

Catalepsy test

Both forepaws of mice are placed on a horizontal bar elevated 15 cm above the ground (0.2 cm diameter). The total time a mouse maintained this position before lifting their hindpaws onto the bar is recorded.

### Swim Test

Mice are placed in water tubs (30 cm×20 cm×20 cm) at a water depth of 12 cm at 27° C. Swim scores are graded as follows: 0, hind part sinks with head floating; 1, occasional swimming using hind limbs while floating on one side; 2, occasional floating/swimming only; and 3, continuous swimming.

### Results

In comparison to the saline vehicle control group, the MPTP treated group exhibits poorer scoring in all three tests (longer time to descend, longer time to lift hind paws and lower swim test score). The KLK1+MPTP treated group exhibits statistically significant better scores in all three tests separately compared to the MPTP treated group. These results suggest that KLK1 attenuates behavioral impairments caused by MPTP, a neurodegeneration inducing neurotoxin.

### Example 5

### KLK1 Prevents the Reduction of Spontaneous Locomotion Observed in Truncated Human α-Synuclein (1-120) Transgenic Mouse

Transgenic mice expressing truncated human α-Synuclein (1-120) in an α-Syn null background under the TH promoter (Tofaris et al., J Neurosci. 2006, 26:3942-50) display the formation of Lewy bodies made of α-Syn in the SNc, much like the α-synucleinopathy seen in PD.

### Spontaneous Locomotor Activity Testing

Spontaneous locomotor activity (LMA) levels of the 18 month old transgenic mice compared to 18 month wild type mice are tested. The 18 month transgenic mice are subdivided into two groups of 20 each, in which one group is treated with KLK1 intranasally for eight weeks daily prior the start of LMA while the other is left untreated.

LMA testing is performed in clear Perspex® box (210×210×365 mm) cages fitted with two transverse infrared beams 10 mm from the base, spaced equally along the length of the box. The number of beam breaks during the 30 min session is recorded by a computer.

### Results

The 18 month old, non-treated transgenic mice show a statistically significant decrease in the number of infrared beam breaks compared to 18 month old wild-type mice. The 18 month old KLK1 treated transgenic mice show a significant increase in the number of infrared beam breaks compared to the 18 month non-treated transgenic group. This result suggests that treatment of transgenic mice expressing truncated α-Syn with KLK1 prevents the α-Syn mediated loss of locomotion as similarly seen in PD.

1. A method of treating Parkinson's disease or an associated condition comprising administering tissue kallikrein, or a variant or active fragment thereof, wherein the variant or active fragment has at least 80% sequence identity to tissue kallikrein and serine kinase activity.

2. (canceled)

3. The method of claim 1, further comprising administering an additional Parkinson's disease therapeutic compound.

4. The method of claim 3, wherein administering the additional Parkinson's disease therapeutic compound is concurrent with the administering of tissue kallikrein, or a variant or active fragment thereof.

5. A method of treating dementia with Lewy bodies or an associated condition comprising administering tissue kallikrein, or a variant or active fragment thereof, wherein the variant or active fragment has at least 80% sequence identity to tissue kallikrein and serine kinase activity.

6. (canceled)

7. A method of treating a disease or symptom selected from the group consisting of synucleinopathy, parkinson-plus syndrome, dementia, elirium, visual hallucination parkinsonism, and depression, comprising administering tissue kallikrein or a variant or active fragment thereof, wherein the variant or active fragment has at least 80% sequence identity thereto and having serine kinase activity.

8. (canceled)

9. (canceled)

10. A composition comprising tissue kallikrein and a compound selected from the group consisting of a pharmaceutically acceptable carrier for intranasal administration and a propellant for intranasal administration.

11. A composition comprising tissue kallikrein and an additional Parkinson's Disease therapeutic compound.

12. The composition of claim 11 wherein the additional Parkinson's Disease therapeutic compound is selected from the group consisting of an anticholinergic agent, an antiinfective agent, a catechol-O-methyl (COMT) transferase, a dopamine agonist, a monoamine oxidase type B (MAO-B) inhibitor, a neurological agent, a nutritional supplement, a psychotrophic agent, and an antidepressant.

13. The composition of claim 12 wherein the antiinfective agent is amantadine.

14. The composition of claim 12 wherein the COMT transferase is selected from the group consisting of carbidopa, entacapone, levodopa, and tolcapone.

15. The composition of claim 12 wherein the dopamine agonist is selected from the group consisting of apomorphine, bromocriptine, cabergoline, pergolide, pramipexole, and ropinirole.

16. The composition of claim 12 wherein the MAO-B inhibitor is selected from the group consisting of rasagiline and selegiline.

17. The composition of claim 12 wherein the neurological agent is selected from the group consisting of brasofensine, istradefylline, and leteprinim.

18. The composition of claim 12 wherein the nutritional supplement is selected from the group consisting of coenzyme Q-IO, ubiquinone, and creatine.

19. The composition of claim 12 wherein the psychotrophic agent is diphenhydramine.

20. The composition of claim 12 wherein the antidepressant is selected from the group consisting of a selective serotonin reuptake inhibitors, a tricyclic antidepressant, mirtazapine, moclobemide, phenelzine, and venlafaxine.

21. The method of claim 1 further comprising deep brain stimulation.

22. (canceled)

23. The method of claim 1 wherein the tissue kallikrein, or variant or active fragment thereof, is administered as an oral dosage of 0.001 to 5000 IU.

24. The method of claim 1 wherein the tissue kallikrein, or variant or active fragment thereof, is administered as an intranasal dosage of 0.001 to 5000 IU.


SEQUENCE LISTING (2 sequences)

SEQ ID NO: 1 - protein, 263 aa, Sus scrofa
MWSLVMRLALSLAGTGAAPPIQSRIIGGRECEKDSHPWQVAIYHYSSFQCGGVLVDPKWVLTAAHCKNDNYQVWLGRHNLFENEVTAQFFGVTADFPHPGFNLSLLKNHTKADGKDYSHDLMLLRLQSPAKITDAVKVLELPTQEPELGSTCQASGWGSIEPGPDDFEFPDEIQCVELTLLQNTFCADAHPDKVTESMLCAGYLPGGKDTCMGDSGGPLICNGMWQGITSWGHTPCGSANKPSIYTKLIFYLDWINDTITENP

SEQ ID NO: 2 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS